(19) United States
(12) Patent Application Publication
(10) Pub. No.: US 2026/0207996 A1
(43) Pub. Date: Jul. 23, 2026

(54) Athletic assessment device and system

(71) Applicant: Rabboni Co., LTD., Hsinchu City (TW)

(72) Inventor: Kuei Ann WEN, Hsinchu County (TW)

(21) Appl. No.: 19/353,371

(22) Filed: Oct. 8, 2025

(30) Foreign Application Priority Data
Jan. 17, 2025 (TW) 114102156

(51) Int. Cl. A63B 24/00

(52) U.S. Cl. CPC A63B 24/0062; A63B 2024/0065; A63B 2220/30; A63B 2220/51; A63B 2220/836; A63B 2225/20

(57) ABSTRACT

Athletic assessment device and system include a plurality of inertial sensors and a physical/sports fitness assessment device that includes at least one physical fitness component calculator, each calculating one of balance, agility, explosive power, speed and flexibility, all relating to physical fitness status of an object. The output data of the athletic assessment device and system include data indicating one of said physical fitness components.

### TECHNICAL FIELD

The present invention relates to athletic assessment device and system, and in particular to a device and system that can assess multiple athletic actions using a single detection device.

### BACKGROUND OF THE INVENTION

Inertial sensors and other types of displacement sensors based on microsystem engineering technology are widely used in wearable devices, for athletic assessment. In this technical field, various devices that use displacement sensors in combination with other sensors or sensing technologies, including image capture devices, to achieve convenient and accurate athletic assessment are introduced.

US20110281249A1 proposes a method and system for creating personalized workout programs. The method and system are used to evaluate and adjust a physical training program and include the steps of:
receiving first input data from a client device, which data comprise personal fitness goal information and scheduling data. Based on the first input data, a recommended workout schedule and a recommended list of activity groups are generated. Receiving second input data from the client device, which data comprising feedback related to the recommended list of activity groups. And generating a personalized workout plan, based on the first and second input data. The personalized workout plan may be displayed on the client device.

U.S. Pat. No. 8,503,086B2 discloses a system and method for tracking and assessing movement skills in multidimensional space. The invention proposes a multiuser physical activity system comprising: at least one tracking system for determining changes in general physical locations of a first user and a second user; and at least one processor operatively coupled to the at least one tracking system for updating user virtual locations in a virtual space, corresponding to the physical locations of the users. The at least one tracking system is an image capture device or a wireless optical sensor.

US20180021627A1 discloses an interactive and dynamic fitness system Interactive and Dynamic Fitness System. The system detects the training activities that the trainee has engaged in or the training equipment that has been used based on the trainee's dynamic position.

US20230024272A1 discloses an athletic skills development ranking and tracking method. This invention detects the performance of athletes and provides assessments. This assessment is the result of comparing scores with professional standards. The invention, however, is limited to sports performance in shooting training.

U.S. Ser. No. 11/364,418B2 relates to a device, system and method for automated global athletic assessment and/or human performance testing. The method collects and analyzes collect and analyze the physical performance of an individual and structure and generate an athletic protocol. Multiple athletic assessment devices perform an athletic assessment of an individual, including kinetic, neurological, musculoskeletal and aerobic capacity. More specifically, the assessment includes body composition, orthopedic/musculoskeletal assessment, biomechanical, neurological/sensorimotor skills, aerobic/anaerobic, and/or capacity assessments and testing.

WO2006074297A2 discloses a system, method and apparatus for evaluating military personnel. The invention also uses a plurality of specific purpose measuring instruments to detect and evaluate athletic movements.

CN105705093A relates to conformal sensor systems for sensing and analysis. According to the invention, flexible electronics technology are implemented as conformal sensors for sensing or measuring motion (including body motion and/or muscle activity), heart rate, electrical activity, and/or body temperature for such applications as medical diagnosis, medical treatment, physical activity, physical therapy and/or clinical purposes. The embodiment further compares the measurement results of the two sides of the human body to calculate a balance value.

JP6210997B2 discloses a method and system for automated personal training, including training programs, for creating a personalized exercise plan. An image capture device and a computer device are used to capture images of the user while the user performs a physical activity to create a human motion test score based on an evaluation of the resulting images. A personalized exercise program that identifies the user's body area for improvement based on the one or more human motion screening scores is generated.

JP2021137415A discloses wearable device and muscle function parameter calculation system. The patent calculates muscle function parameters indicating a muscle function of a user. A wearable device having an acceleration sensor whose detectable acceleration range is wider than ±20 G and narrower than ±40 G is used, which sensing data are used to calculate a muscle function parameter indicating the level of a muscle function of a user. The sensor used may be a wearable device.

U.S. Ser. No. 11/298,036B2 discloses a wearable device including PPG and inertial sensors for assessing physical activity and biometric parameters. A plurality of PPG waveforms is collected from a PPG sensor and inertial data associated with subject motion are collected from an inertial sensor. A neural network is used to estimate the subject's blood pressure based on multiple PPG waveforms.

EP3058442B1 discloses calculating pace and energy expenditure from athletic movement attributes. The invention collects kinematic data from an activity monitoring device during initial activity to build an expenditure model. The participant's current kinematic data are collected during exercise, processed according to the expenditure model to monitor expenditure status, which status is displayed on the user interface.

US20190183412A1 discloses a system and method for assisting exercising of a subject. An exercise state providing unit provides the exercise state of the object for or during exercise; a fatigue level determining unit determines the fatigue level of the object. An evaluation unit issues a warning signal when the fatigue level is higher than a threshold.

U.S. Ser. No. 11/497,966B2 discloses an automatic coaching system and method for coaching user's exercise. The invention calculates a metric using data collected by an acceleration sensor or a location sensor worn by the user, selects a new coaching target metric based on an error rate, and outputs a coaching message related to the coaching target metric

TWI796035B filed by the same inventor discloses a biomechanics assessment system and biomechanical sensing device and biomechanical assessment platform thereof. The biomechanical information interpretation device is equipped with a biomechanical information interpretation program. After operation, the biomechanical information obtained by the biomechanical sensing device can be sued to mark features, mark reference information, for example, type of movement type, type of actions, position of the sensor, sensing time, stage of a sensing history; and normalizes the biomechanical data so obtained.

US20240091589A1 relates to an integrated portable device and method implementing an accelerometer for analyzing biomechanical parameters of a stride. When a runner runs a certain distance (D) along a running course, having a processor calculate the biomechanical parameters of the stride, including the lowering (A) of the center of gravity, the elevation (E) of the center of gravity, the sum of the lowering and of the elevation of the center of gravity and the vertical mechanical work of the center of gravity (CG) of said runner, so to measure a sequence of acceleration data in at least the vertical direction (av) using said accelerometer.

U.S. Pat. No. 8,626,472B2 discloses a system and method for measuring balance and track motion in mammals. A sensor band attached to the mammal's calf and used to measure the track and balance motion of the mammal, comprising one or more first sensors for sensing muscle circumferential pressure at multiple calf positions; one or more second sensors for sensing the Earth's magnetic field; and one or more third sensors for sensing the Earth's gravity field.

US20070219059A1 relates to method and system for continuous monitoring and training of exercise. The system comprises a multi-sensor data acquisition system to measure body sounds, body signs, vital signs, motions, and machine settings continuously and automatically. The system is capable of capturing capture the body sounds and other vital signs, analyze them, and report and display summarized results. The signal processing functions utilize a unique signal separation and noise removal methodology by which authentic signals can be extracted from interfered signals and in noisy environments, even when signals and noises have similar frequency components or are statistically dependent.

KR101651429B1 discloses a fitness monitoring system. When the operation of a person performing the reference operation is displayed on the recording medium and the user performs the corresponding operation, the type, intensity, frequency and accuracy of the corresponding operation are determined and displayed. The motion recognition devices used include: pulse sensors; accelerometers; and gyroscopes, which provide body rotation information and direction information in response to the user's movements.

KR20220106448A reveals a personal exercise management system using artificial intelligence and electromyographic signa, which combines artificial intelligence and wireless myoelectric signal processing to determine a movable range of fitness and Taekwondo movements. When performing functional movements, the coordination or co-contraction of nerves and muscles of the muscle groups involved in the movement may be improved.

US20240315390A1 discloses instrumented artificial intelligence (AI) driven motion tracking and alignment systems and methods for various applications. The invention generates generate time-normalized three-dimensional (e.g. frontal, sagittal, transverse) data associated with pronation and/or supination of lower extremities of a user. A semi-rigid foot orthotic can have sensors embedded in flexible regions. Data from the sensors is used to measure how the orthotic bends to determine forces from a lower extremity of the user acting on the orthotic. Data on the flexing, bending, and/or rotating of portions of the orthotic (including velocities, accelerations) may be compared to analyze in-shoe pronation and/or supination.

It can be known from these prior art documents that the industry is currently in urgent need of devices and systems that can evaluate athletic performance during exercise. The device should be wearable by athletes without hindering exercise and can collect sufficient and useful information for athletic assessments. Currently proposed athletic assessments devices and systems are mainly based on inert sensors such as accelerometers and gyroscopes, but they must be supplemented by a variety or even a large number of other detection devices to provide the required sensing data.

In addition, although most existing technologies can provide accurate athletic assessment, they can only evaluate a single or a few types of sports. A few systems can provide evaluation for multiple components or features of an athletic movement, but the same set of athletic assessment devices and systems can only conduct athletic assessments for a single part of the body or a single athletic item or action. The few tools that can assess multiple parts or more than one sport item are limited to the particular parts, items or the like. In addition, traditional athletic assessments must combine motion-related sensing devices and at least one other sensing device, such as physiological detection devices, image capture devices, etc. Inconvenience is felt when wearing, exercising or processing data.

### SUMMARY OF THE INVENTION

The objective of the present invention is to provide an athletic assessment device and system that can use a single sensing device to evaluate multiple sports.

In particular, an object of the present invention is to provide an athletic assessment device and system that can serve their assessment functions for multiple sports using only a single sensing device.

The present invention also aims to provide an athletic assessment device and system with scalable assessment methods and/or sports items.

The present invention also aims to provide an athletic assessment device and system that can adjust assessment methods and/or assessable items according to the needs of trainees.

The athletic assessment device according to the present invention comprises at least one inertial sensing device and a computing device. In a preferred embodiment of the present invention, the inertial sensing device can be a wearable device, used to be worn on the human body or limbs, and to sense the motions of the sensing device itself when the human body is in exercise. The sensing results that the inertial sensing device generates may include acceleration, angular velocity, magnetism, or any combination thereof. The inertial sensing device preferably comprises a set of plural inertial sensors, and the inertial sensors are preferably nine-axis inertial sensors. The inertial sensing device communicates with the computing device in a wired or wireless manner. In order to provide a wireless communication connection, the inertial sensing device is equipped with a wireless communication module, preferably a short-range wireless communication module, and the computing device is also equipped with a corresponding wireless communication module, preferably a short-range wireless communication module, to establish a communication channel between the two. When using the wired communication channel, it is preferably a pluggable communication channel. To this end, the inertial sensing device can be equipped with a communication slot for plugging and unplugging the communication line equipped with the computing device. The inertial sensing device also provides a power module to power the inertial sensor and the communication module. The power module shall include a power storage device.

The inertial sensing device is further equipped with a controller for receiving control parameters provided by the computing device and using the control parameters to establish or update the setting value of the inertial sensing device to regulate the output data of the inertial sensor. The parameters can be used to regulate, such as, the type, format, scale of values, resolution, output frequency and other parameters of the output data. The computing device is equipped with a physical fitness measurement device (or sports fitness measurement device), comprising a plurality of physical fitness calculators, each of which calculates a value of at least one element of physical fitness. In a preferred embodiment of the present invention, the physical fitness calculation functions provided by the fitness calculation device, that is, the calculated physical fitness elements include at least one of the following items: balance, agility, power, speed, and flexibility. In other embodiments the calculated elements of fitness may also include coordination, reaction, etc.

The computing device also includes an input/output data controller connected to the wireless communication module or a wired communication module to receive the output data of the inertial sensor from the inertial sensing device and provide them to the physical fitness measurement device, and to receive the calculation results of the physical fitness measurement device, i.e., physical fitness element related sensing results, which are always a value of a corresponding physical fitness element, and provide the calculation results to the outside world through an output interface, or display them on the display device of the computing device.

The computing device may further comprise an athletic assessment device including a storage device for storing at least one athletic assessment application. After the athletic assessment application is executed in the computing device, an athletic assessment item can be calculated based on at least one of the physical fitness element related sensing results.

In a preferred embodiment of the present invention, the physical fitness calculators can calculate the value of corresponding physical fitness elements according to the following formulae:

For calculating balance:

$$Balance = {Σ}_{k = x , y , z} \vert \vert {ACC}_{k} ( t ) \vert - \vert {ACC}_{k} ( t - 1 ) ‖ + ‖ {GYRO}_{k} ( t ) \vert - \vert {GYRO}_{k} ( t - 1 ) ‖$$

To calculate Agility:

$$Agility = \frac{\int \begin{matrix}{t}_{cycle end} \\ {t}_{cycle start}\end{matrix} {Vel}_{lateral} ( τ ) d τ - drift error}{{t}_{cycle end} - {t}_{cycle start}} ( Sensor put on target position )$$

$$wherein , {Vel}_{lateral} ( τ ) = f {\sum }_{k = x , y , z} \vert ACCk ( n ) \vert dn$$

$$Power = F \times V = Force \times velocity$$

$$wherein , F = m \times \sqrt{{a}_{x}^{2} + {a}_{y}^{2} + {a}_{z}^{2}}$$

$${v}_{x} [ k ] = {v}_{x} [ k - 1 ] + {a}_{x} [ k ] \times Δ t$$

$${v}_{y} [ k ] = {v}_{y} [ k - 1 ] + {a}_{y} [ k ] \times Δ t$$

$${v}_{z} [ k ] = {v}_{z} [ k - 1 ] + {a}_{z} [ k ] \times Δ t$$

$$V = \sqrt{{{v}_{x} [ k ]}^{2} + {{v}_{y} [ k ]}^{2} + {{v}_{z} [ k ]}^{2}}$$

When calculating the speed:

$$Speed = {v}_{x} [ k ] = {v}_{x} [ k - 1 ] + {a}_{x} [ k ] \times Δ t$$

$${v}_{y} [ k ] = {v}_{y} [ k - 1 ] + {a}_{y} [ k ] \times Δ t$$

$${v}_{z} [ k ] = {v}_{z} [ k - 1 ] + {a}_{z} [ k ] \times Δ t$$

$$V = \sqrt{{{v}_{x} [ k ]}^{2} + {{v}_{y} [ k ]}^{2} + {{v}_{z} [ k ]}^{2}}$$

When calculating Flexibility of a human body:

$$Flexibility = {\sum }_{k = x , y , z} \int {GYRO}_{k}^{2} ( t ) dt .$$

The present invention also provides an athletic assessment system, comprising a plurality of any one of the athletic assessment devices of the present invention, and at least one athletic assessment server, which is communicatively connected to the plurality of athletic assessment devices in a wired and/or wireless manner. The athletic assessment server is equipped with an athletic assessment knowledgebase for storing the physical fitness element related sensing results and/or athletic assessment results sent by the plurality of athletic assessment devices. The athletic assessment server can also provide various athletic assessment applications, as well as a computing device. After an athletic assessment application is executed in the computing device, an athletic assessment item can be calculated according to at least one of the physical fitness element related sensing results. The athletic assessment server can also establish a variety of general athletic assessment applications for performing a general athletic assessment based on a single kind or multiple kinds of physical fitness element related sensing results and/or athletic assessment results sent by a plurality of athletic assessment devices. The athletic assessment server can also respond to a request from at least one athletic assessment device to provide stored physical fitness element related sensing results and/or athletic assessment results and/or general athletic assessment values.

The above and other objectives and advantages of the present invention will become clearer from the following detailed description, with reference to the accompanying drawings.

### BRIEF DESCRIPTION OF THE DRAWINGS

FIG. 1 shows a block diagram of an embodiment of the athletic assessment system of the present invention.

FIG. 2 shows a block diagram of a preferred embodiment of the athletic assessment device used in the athletic assessment system of the present invention.

### DETAILED DESCRIPTION OF THE INVENTION

Several preferred embodiments of the athletic assessment device and system of the present invention are described below with reference to the drawings. However, the purpose of the embodiments is to enable the skilled persons to implement the methods, devices and systems of the present invention after reading the patent specification. The embodiments are only various possible implementations of the present invention, and are not intended to exhaustively illustrate all aspects and applications of the present invention. The scope of the invention should only be defined by the attached claims.

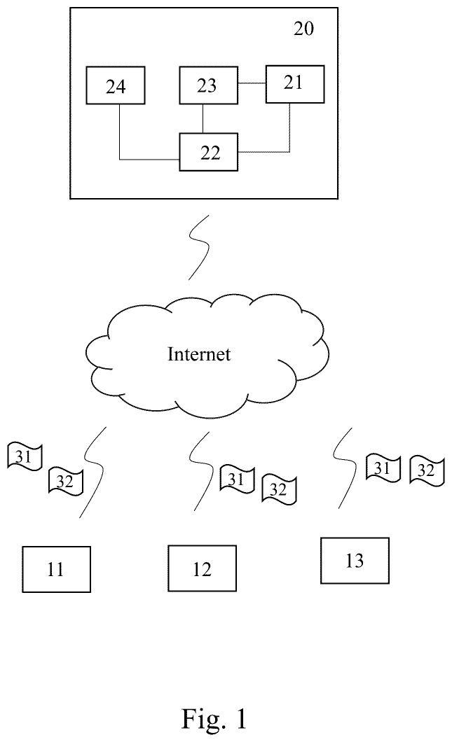

FIG. 1 shows the block diagram of an embodiment of the athletic assessment system of the present invention. As shown in the figure, the athletic assessment system of the present invention includes a plurality of any athletic assessment devices 11, 12, 13 of the present invention, and at least one athletic assessment server 20. The athletic assessment server 20 is communicatively connected to the plurality of athletic assessment devices 11, 12, 13 in a wired and/or wireless manner. As shown in the figure, the athletic assessment server 20 is equipped with a computing device 22 and an athletic assessment knowledgebase 21. The athletic assessment knowledgebase 21 can store the physical fitness element related sensing results 31 and/or sports assessment results 32 provided by the plurality of athletic assessment devices 11, 12, 13. The athletic assessment server 20 can also store a variety of athletic assessment applications 23. After the exercise assessment application program 23 is executed in the computing device 22, it can calculate an exercise assessment project result based on at least one of the physical fitness element related sensing values 31. The athletic assessment server 20 can also embed a variety of general athletic assessment applications 24 for calculating an general athletic assessment result using one or a plurality of physical fitness element related sensing values 31 and/or sports assessment results 32 provided by the plurality of athletic assessment devices 11, 12, 13. The athletic assessment server 20 can also provide stored physical fitness element related sensing values and/or sports assessment sensing results and/or general athletic assessment results to at least one athletic assessment device 11, 12, 13 in response to their requests.

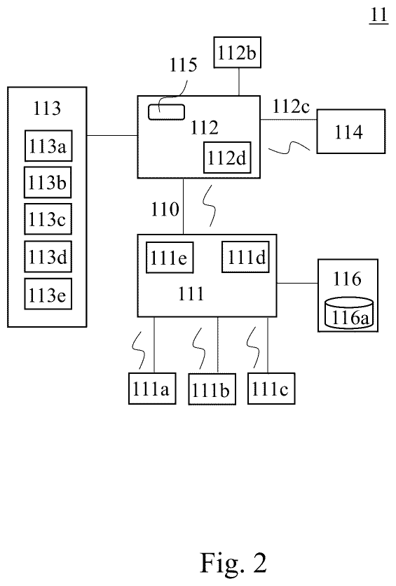

FIG. 2 is a block diagram showing a preferred embodiment of an athletic assessment device suitable for the athletic assessment system of the present invention. As shown in the figure, the athletic assessment device 11, 12, 13 according to the preferred embodiment of the present invention comprises an inertial sensing device 111 and a computing device 112. In a preferred embodiment of the present invention, the inertial sensing device 111 can be formed into a wearable device for wearing on a human body, a limb or other part of the body to sense the motion of the sensing device 111 itself when the human body moves. The sensing results may include acceleration, angular velocity, magnetism, or any combination thereof. The inertial sensing device 111 preferably includes a set of inertial sensors 111a, 111b, 111c. The inertial sensors 111a, 111b, 111c are preferably nine-axis inertial sensors. The inertial sensing device 111 is connected to the computing device 112 in a wired or wireless manner. To provide wireless communication connection, the inertial sensing device 111 is equipped with a wireless communication module 111d, preferably a short-distance wireless communication module, and the computing device 112 is also equipped with a corresponding wireless communication module 112d, preferably a short-distance wireless communication module. The communication modules establish a communication channel between the two. If it is a wired communication channel, it is preferably a pluggable communication channel. To this end, the inertial sensing device 111 may be equipped with a communication slot for plugging and unplugging a communication line 110 equipped by the computing device 112. The inertial sensing device 111 further provides a power module (not shown) to supply power to the inertial sensors 111a, 111b, 111c and the communication module 111d. The power module should include a power storage device.

The athletic assessment device with the above configurations and functions is already a mature product. Commercially available sensing chips can usually provide the sensing functions of a three-axis accelerometer, a three-axis gyroscope, and a three-axis geomagnetometer, as well as short-distance wireless communication functions, such as the Bluetooth-standard communication channels, allowing the sensing chip to transmit the sensing results, or the calculation results thereof, to the outside world wirelessly. Inertial sensing device 111 suitable for the present invention can be produced using the commercially available sensing chips. The computing device 112 can also be established, based on a commercially available processor or microprocessor, supplemented by appropriately designed application software, to perform required processing on the sensing results of the inertial sensing device 111 to obtain the desired results. The computing power of commercially available processors or microprocessor chips is sufficient to provide the computing functions required by the present invention. Detailed description thereof is thus omitted.

In a preferred embodiment of the present invention, the inertial sensing device 111 is further equipped with a controller 111e for receiving control parameters sent from the computing device 112 and using the control parameters to configure or to update the settings of the inertial sensing device 111. The settings are used to regulate the output data of the inertial sensors 111a, 111b, 111c, such as determining the type, format, data value range, output frequency and other parameters of the output data, of the inertial sensors 111a, 111b, 111c. For the establishment of the above-mentioned sensing parameters, etc., in most applications, the firmware provided by the inertial sensing device 111 is sufficient for the above-mentioned application in regulating the output data. However, equipping the controller 111e can improve the control capability or add control functions, or both, to support more complex applications. The inertial sensing device 111 with the above configurations and functions can be manufactured by the skilled persons using commercially available processors or microprocessors as needed. Detailed description thereof is thus omitted.

As one of the important features of the present invention, the computing device 112 is equipped with a physical fitness measurement device (or sports fitness measurement device) 113, including a plurality of physical fitness calculators 113a, 113b, 113c, 113d, 113e, each calculating a value of a physical fitness component. In a preferred embodiment of the present invention, the physical fitness measurement functions provided by the physical fitness measurement device 113, that is, the physical fitness components to which the calculating results of each calculator 113a, 113b, 113c, 113d, 113e relate, include at least one of the following items: balance, agility, explosive power (power), speed and flexibility. In other embodiments coordination, reaction, etc. may also be included. The relevant technical details will be described below.

The computing device 112 also includes an input/output data controller 114 connected to the wireless communication module 112d or a wired communication module 112c to receive the output data of the inertial sensors 111a, 111b, 111c from the inertial sensing device 111, is provided to the physical fitness measurement device 113, and the physical fitness element value of the calculation result received from the physical fitness measurement device 113 is provided to the outside world through an output interface 112b, or displayed on the display 115 of the computing device.

In a preferred embodiment of the present invention, the computing device 111 may further include a sports assessment device 116, including a storage device 116a for storing at least one sports assessment application program. After the sports assessment application is executed in the computing device 112, a sports assessment result can be calculated based on at least one of the physical fitness element related sensing results 31 or the sports assessment results 32. The details will also be described below.

Regarding the physical fitness measurement device 113, in a preferred embodiment of the present invention, the physical fitness calculators 113a, 113b, 113c, 113d, and 113e equipped with the physical fitness measurement device 113 can respectively calculate values of a corresponding physical fitness element:
Balance Calculator 113a: Balance refers to an individual's ability to maintain the projected line of the center of gravity within the range of the support base. Balance can include static balance and dynamic balance. The general theory is that balance is controlled by three different systems: somatosensory system, visual system and vestibular system. In practice, various outcome tools such as the Berg Balance Scale and BESTest are used for evaluation. In providing a sports or physical fitness assessment, a static balance assessment should be sufficient.

According to a preferred embodiment of the present invention, balance value can be calculated according to the following formula, based on the acceleration value and angular velocity value sensed by the inertial sensing device 111:

$$Balance = {\sum }_{k = x , y , z} ‖ {ACC}_{k} ( t ) \vert - \vert {ACC}_{k} ( t - 1 ) ‖ + ‖ {GYRO}_{k} ( t ) \vert - \vert {GYRO}_{k} ( t - 1 ) ‖$$

Wherein, ACCₖ represents the acceleration value measured by the inertial sensor worn on the back waist of the evaluation subject. GYROₖ represents the angular velocity value measured by the inertial sensor worn on the back waist of the evaluation subject. t is the time. Preferably the measurement lasts for 30 seconds, and the offset value during the measurement is calculated. Only one inertial sensing device is used during the measurement. The assessment value is the difference between the values measured by an inertial sensing device before (t−1) and after (t) a certain period of time. The smaller the difference value, the better. Assessors can create their own scales, give scores, and record them, for medium and long-term evaluation.

Agility Calculator 113b: Agility can be defined as “rapid whole-body movements that change speed or direction in response to stimuli.” The human body performs a series of explosive movements in rapid succession in opposite directions when stimulated. The results of measuring these movements can be used to assess agility.

According to a preferred embodiment of the present invention, the agility value can be calculated according to the following formula, based on the acceleration value and angular velocity value sensed by the inertial sensing device 111:

$$Agility = \frac{\int \begin{matrix}{t}_{cycle end} \\ {t}_{cycle start}\end{matrix} {Vel}_{lateral} ( τ ) d τ - drift error}{{t}_{cycle end} - {t}_{cycle start}} ( Sensor put on target position )$$

Wherein, Velₗₐₜₑᵣₐₗ(τ)=∫Σ_{k=x,y,z}|ACCₖ(n)|dn represents the speed value measured by the inertial sensor worn at the position to be evaluated on the body of the evaluation subject. τ is time, which can usually be measured at 0.02 seconds, preferably 0.01 seconds, and optimally 0.005 seconds, all depending on the sampling frequency. A single inertial sensing device is used during measurement. The evaluated value is the value of change in direction within an average time, with the greater the better. The wearing position can be feet, both hands, etc. Evaluators can make their own scales, give scores, and record them, for medium and long-term evaluation.

Explosive power calculator 113c: Explosive power (power) refers to the value of the maximum force a person can exert. In terms of testing, for example, the medicine ball throwing test can be used to evaluate the explosive power of the upper limbs.

According to a preferred embodiment of the present invention, the explosive power can be calculated according to the following formula, based on the three-axis acceleration value sensed by the inertial sensing device 111:

$$Explosive power = F \times V = Force \times velocity$$

In a preferred embodiment of the present invention, the values of F and V can be calculated by the following formula:

$$F = m \times \sqrt{{a}_{x}^{2} + {a}_{y}^{2} + {a}_{z}^{2}}$$

$$V = \sqrt{{{v}_{x} [ k ]}^{2} + {{v}_{y} [ k ]}^{2} + {{v}_{z} [ k ]}^{2}}$$

$$Wherein , {v}_{x} [ k ] = {v}_{x} [ k - 1 ] + {a}_{x} [ k ] \times Δ t$$

$${v}_{y} [ k ] = {v}_{y} [ k - 1 ] + {a}_{y} [ k ] \times Δ t$$

$${v}_{z} [ k ] = {v}_{z} [ k - 1 ] + {a}_{z} [ k ] \times Δ t$$

A single inertial sensing device 111 is used during the test. It is worn on the back waist of the subject. The greater the value, the better. Assessors can create their own scales, give scores, and record them, for medium and long-term evaluation.

Speed calculator 113d: Speed is defined as the ability to complete actions in a short time. Speed can often be measured using sprint testing.

According to a preferred embodiment of the present invention, the speed value can be calculated according to the acceleration value sensed by the inertial sensing device 111 according to the following formula:

$$Speed V = \sqrt{{{v}_{x} [ k ]}^{2} + {{v}_{y} [ k ]}^{2} + {{v}_{z} [ k ]}^{2}}$$

$$Wherein , {v}_{x} [ k ] = {v}_{x} [ k - 1 ] + {a}_{x} [ k ] \times Δ t$$

$${v}_{y} [ k ] = {v}_{y} [ k - 1 ] + {a}_{y} [ k ] \times Δ t$$

$${v}_{z} [ k ] = {v}_{z} [ k - 1 ] + {a}_{z} [ k ] \times Δ t$$

Wherein, a represents the acceleration value measured by the inertial sensors worn on the lower back, hands, feet, etc. of the evaluated subject. The assessment value is based on the sum of the integrated values of the three-axis acceleration. The greater the sum, the better. Assessors can create their own scales, give scores, and record them, for medium and long-term evaluation.

Flexibility Calculator 113e: Flexibility refers to the ability to move a joint through its entire range of motion. Factors that influence flexibility include joint capsule distensibility, adequate warm-up, and muscle viscosity. Common equipment used to evaluate flexibility includes goniometer, inclinometer, Leighton flexometer, etc. However, according to a preferred embodiment of the present invention, the flexibility value can be calculated according to the following formula, based on the acceleration value and angular velocity value sensed by the inertial sensing device 111:

$$Flexibility = {\sum }_{k = x , y , z} \int {GYRO}_{k}^{2} ( t ) dt$$

wherein, GYROₖ represents the angular velocity value measured by the inertial sensor worn on the evaluation subject's hands, feet, waist and other measurement parts. dt is the unit time, usually the bending action is measured several times. Use a single inertial sensing device 111 and wear it on the area to be evaluated. For example, if the flexibility of the waist will be assessed, the inertial sensing device is wore on the waist. If wearing it on the hand, the rotational flexibility of the wrist can be measured. The evaluated value is the integral value of angular velocity, which represents the value of angular in orientation. The greater the value, the better. Evaluators can make their own scales, give scores, and record them, for medium and long-term evaluation.

Although it is academically believed that only the flexibility of specific joints can be measured and the flexibility of the whole body cannot be measured, the flexibility of the whole body or specific sections of the trunk can be measured using the technology of the present invention.

### EMBODIMENTS

In order to verify that the athletic assessment device and system of the present invention can indeed be used to assess the various elements of human body fitness, the following experiments were conducted.

### Embodiment 1—Assessment of Flexibility 1—Sitting Forward Bend Test

An inertial sensor according to an embodiment of the present invention is worn on the user's back waist. Have the user sit on the floor with the legs stretched forward. Stretch both arms forward. Turn on the computing device and the inertial sensing device, and pair the frequencies so that the two can establish a communication connection. Turn on the flexibility assessment function 1 of the computing device.

After starting the measurement, ask the user to bend the body forward and keep the arms extended forward. Return to original position and bend forward. Repeat this 10 times. Record each waist bending angle measured by the inertial sensing device during this period.

Determining the direction of movement: The current calculation formula is not directional. The square root of the change angles in the three directions is added together as the total change angle.

Determination of bending angle: By integrating the angular velocity, the bending angle during the process can be obtained.

Eliminate invalid single movements, such as test results with too small a bending angle. Calculate the average of the bending angles.

### Embodiment 2—Assessment of Flexibility 2—Thigh Rotation Angle Measurement

The athletic assessment device of the present invention is used to assess human body flexibility, by performing the following test.

An inertial sensor according to an embodiment of the present invention is worn on the user's left ankle. Have the user lie on the back. Turn on the computing device and the inertial sensing device, and pair the frequencies so that the two can establish a communication connection. Turn on the flexibility assessment function 2 of the computing device.

When starting the detection, measure the starting angle. Have the user to straighten and lift the left leg, reach the maximum amount of stretch and then return to the original position. Repeat this 10 times. Record each single-directional movement/rotation angle measured by the inertial sensing device during this period.

To determine the starting direction, the user first remains still, then use the gravity acceleration to determine the starting direction of the sensor.

To determine the direction of rotation, the calculation formula is not directional. The square root of the change angles in the three directions is added together to produces the total change angle.

To determine the rotational angle, the angular velocity is integrated, to obtain the angle of rotation during the process.

Eliminate invalid single movements, such as test results with too small angles. Calculate the average of the angle changes.

### Embodiment 3—Assessment of Explosive Power—Vertical Jump Measurement

The athletic assessment device of the present invention is used to assess the explosive power of the human body, by performing the following test.

An inertial sensor according to an embodiment of the present invention is worn on the user's right wrist. The user is asked to stand with the feet shoulder-width apart, the feet slightly bent, the heels on the ground, and the hands on the waist. Turn on the computing device and the inertial sensing device, and pair the frequencies so that the two can establish a communication connection. Turn on the explosive power assessment function of the computing device.

After starting the test, the user is asked to squat and jump to the ground, and then land naturally after reaching the maximum amount of activity. During this period, both hands remain on the waist. Repeat this 10 times. Record each single-directional acceleration measured by the inertial sensing device during this period. According to the principle of F=m×a, the force, speed and flight time of each movement are calculated.

Determination of moving direction: Use the positive and negative values of the sensor to determine the direction.

Calculation of force: Use the measured acceleration to calculate the force, after excluding individual differences in each person's weight.

Calculation of speed: Calculate by dividing the jumping distance by the time.

Calculation of flight time: Use the sampling rate of the sensor to calculate the corresponding time. The beginning and the end of the flight can be judged by the acceleration waveform.

Eliminate invalid single movements, such as test results that are too short, for example, a value that does not exceed a threshold or exceeds a threshold. Calculate the average value of the test items.

### Example 4—Assessment of Balance—Static Balance Test

The athletic assessment device of the present invention is used to assess human body balance, by performing the following test.

A sensor according to an embodiment of the present invention is worn on the user's lower back. Let the user stand naturally, with feet together, heels on the ground, and hands hanging down naturally. Turn on the computing device and the inertial sensing device, and pair the frequencies so that the two can establish a communication connection. Turn on the balance assessment function of the computing device.

After starting the test, ask the user to 1) keep standing with eyes open for 30 seconds. 2) Maintain standing position with eyes closed for 30 seconds. 3) Open the eyes and lift one foot back, maintaining this position for 30 seconds. Do this once on each foot. 4) Close the eyes and lift one foot back, maintaining this position for 30 seconds. Do this once on each foot. 5) Open the eyes and move a foot forward, till the toes of the back foot touching the heel of the front foot, and maintain standing for 30 seconds. Swap the feet and measure again. 6) Close the eyes and move one foot forward, till the toes of the back foot touching the heel of the front foot, and maintain standing for 30 seconds. Swap the feet and measure again. Measure the amount of body sway during the test.

The formula for determining the degree of swaying:

$$Average change of ACC in R axis : \sum ( \vert {Acc}_{Rn} - {Acc}_{Rn + 1} \vert ) / N °$$

$$Average change of GY in R axis : \sum ( \vert {GY}_{Rn} - {GY}_{Rn + 1} \vert ) / N °$$

Eliminate invalid single movements and eliminate data that is too different from other data. Calculate the average value of the test items.

### Example 5—Assessment in Reaction Time—Simple Response and Selected Response Testing

The athletic assessment device of the present invention is used to assess human body reaction time, by performing the following test.

An initial sensor according to an embodiment of the present invention is worn on the user's dominant wrist. Have the user sit on a chair with his hands naturally placed on the table in front of him. Turn on the computing device and the inertial sensing device, and pair the frequencies so that the two can establish a communication connection. Turn on the response time assessment function of the computing device.

After starting the detection, place the display of a computer, such as the screen of a smartphone, on the table in front of the user and start the application. The simple response test requires users to raise their hands when the light pattern on the mobile phone screen lights up. Then let it go. The selected response test requires the user to reach out in different directions according to the graphics displayed on the mobile phone screen. Repeat this 10 times. The time between each graphic display point and the time when the wrist movement distance in a single direction reaches the threshold, measured by the inertial sensing device, is recorded.

The threshold can be a value determined after experimental testing. The test only needs to measure the time when the threshold is reached. Eliminate invalid single movements, such as test results with too short distances and too long reaction times. Calculate the average of reaction times.

As mentioned above, the inertial sensing device 111 in the embodiment of the present invention may be further equipped with a controller 111e, which is used to receive control parameters sent from the computing device 112 and to use the control parameters to configurate or update the settings of the inertial sensing device 111, to regulate the output data of the inertial sensors 111a, 111b, 111c, such as in the type, format, data value range, output frequency and other parameters of the output data. In most embodiments, the inertial sensing device 111 does not directly output the sensing result data (raw data), or does not only output the sensing result data, but outputs the values of the aforementioned physical fitness elements.

Professionals in the application fields of sports assessment, health assessment, medical assessment, learning assessment, etc. do not need to understand the sensing value characteristics of various sensors, such as accelerometers, angular velocity meters, magnetometers, etc., nor do they need to purchase, and ask the subjects to wear and replace sensors specifically designed for each single elements of physical fitness. By using the invented physical fitness measurement device, they can assess physical fitness of a subject by using the output data of the present invention or add value to the assessment results.

For example, in the aforementioned embodiment of explosive power assessment, the inertial sensing device 111 may be configured to additionally output an assessment value of the balance degree. This allows the subject's balance to be continuously monitored while assessing his/her explosive power.

The present invention provides a novel athletic assessment device and system, which enable experts in various industries to easily apply the athletic assessment device to different tests and combine different tests to develop various applications without having to understand the various sensors or the nature of the sensing data.

1. An athletic assessment device, comprising at least one inertial sensing device and a computing device, wherein the inertial sensing device comprises a plurality of inertial sensors that are communicatively connected to the computing device in a wired or wireless manner; wherein the inertial sensing device is equipped with a controller for accepting control parameters sent by the computing device, and using the control parameters to configurate or update a setting value of the inertial sensing device, thereby regulating an output data of the displacement sensor; wherein, the computing device is equipped with a physical fitness measurement device, comprising a plurality of physical fitness calculators, individually calculating a value of at least one of the following components of physical fitness: Balance, agility, explosive power, speed, and flexibility; and wherein an output data of the computing device comprises an assessed value of at least one of the physical fitness elements.

2. The athletic assessment device of claim 1, wherein the physical fitness calculator calculates the balance value according to the following formula:
$$Balance = {\sum }_{k = x , y , z} ‖ {ACC}_{k} ( t ) \vert - \vert {ACC}_{k} ( t - 1 ) ‖ + ‖ {GYRO}_{k} ( t ) \vert - \vert {GYRO}_{k} ( t - 1 ) ‖$$

3. The athletic assessment device of claim 1, wherein the physical fitness calculator calculates the agility value according to the following formula:
$$Agility = \frac{\int \begin{matrix}{t}_{cycle end} \\ {t}_{cycle start}\end{matrix} {Vel}_{lateral} ( τ ) d τ - drift error}{{t}_{cycle end} - {t}_{cycle start}} ( Sensor put on target position )$$
$$wherein {Vel}_{lateral} ( τ ) = f {\sum }_{k = x , y , z} \vert ACCk ( n ) \vert dn .$$

4. The athletic assessment device of claim 1, wherein the physical fitness calculator calculates the value of explosive power according to the following formula:
$$Explosive power = F \times V = Force \times velocity$$
$$wherein F = m \times \sqrt{{a}_{x}^{2} + {a}_{y}^{2} + {a}_{z}^{2}}$$
$$V = \sqrt{{{v}_{x} [ k ]}^{2} + {{v}_{y} [ k ]}^{2} + {{v}_{z} [ k ]}^{2}}$$
$${v}_{x} [ k ] = {v}_{x} [ k - 1 ] + {a}_{x} [ k ] \times Δ t$$
$${v}_{y} [ k ] = {v}_{y} [ k - 1 ] + {a}_{y} [ k ] \times Δ t$$
$${v}_{z} [ k ] = {v}_{z} [ k - 1 ] + {a}_{z} [ k ] \times Δ t .$$

5. The athletic assessment device of claim 1, wherein the physical fitness calculator calculates the value of speed according to the following formula:
$$Speed V = \sqrt{{{v}_{x} [ k ]}^{2} + {{v}_{y} [ k ]}^{2} + {{v}_{z} [ k ]}^{2}}$$
$$in , {v}_{x} [ k ] = {v}_{x} [ k - 1 ] + {a}_{x} [ k ] \times Δ t$$
$${v}_{y} [ k ] = {v}_{y} [ k - 1 ] + {a}_{y} [ k ] \times Δ t$$
$${v}_{z} [ k ] = {v}_{z} [ k - 1 ] + {a}_{z} [ k ] \times Δ t .$$

6. The athletic assessment device of claim 1, wherein the physical fitness calculator calculates the flexibility value according to the following formula:
$$Flexibility = {\sum }_{k = x , y , z} \int {GYRO}_{k}^{2} ( t ) dt .$$

7. The athletic assessment device of claim 1, wherein the inertial sensor is a nine-axis inertial sensor.

8. The athletic assessment device of claim 7, wherein the nine-axis inertial sensor senses acceleration, angular velocity, magnetism or any combination thereof.

9. The athletic assessment device of claim 1, wherein the inertial sensor is a wearable device.

10. The athletic assessment device of claim 1, wherein the control parameter configurates or updates the setting value of the inertial sensing device to regulate the type, format, data value range, output frequency, and maximum value of output data of the inertial sensor.

11. The athletic assessment device of claim 1, wherein the computing device further comprises an athletic assessment device, comprising a storage device for storing at least one athletic assessment application program; wherein after the athletic assessment application program is executed in the computing device, the computing device calculates a value of an athletic fitness element according to at least one of a physical fitness element related sensing result.

12. An athletic assessment system, comprising a plurality of athletic assessment devices as described in claim 1, and at least one athletic assessment server connected to the plurality of athletic assessment devices by wired and/or wireless communication channels; wherein the athletic assessment server is equipped with an athletic assessment knowledgebase for storing the physical fitness element related sensing results and/or athletic assessment results sent by the plurality of athletic assessment devices; wherein the athletic assessment server provides a plurality of athletic assessment applications, which calculate an athletic assessment result according to at least one of the physical fitness element related sensing results, after being executed in the server.

13. The athletic assessment system of claim 12, wherein the athletic assessment server further provides a plurality of general athletic assessment applications for evaluating a single type or a plurality of physical fitness values according to the one or more physical fitness element values and/or physical assessment related sensing results provided by the plurality of athletic assessment devices; and wherein the athletic assessment server is further configured to provide a stored physical fitness element related sensing result and/or an athletic assessment result and/or an general athletic assessment values s, in response to a request from at least one athletic assessment device.
